(19) United States
(12) Patent Application Publication
(10) Pub. No.: US 2004/0215104 A1
(43) Pub. Date: Oct. 28, 2004

(54) Breast exudate expression device and method

(76) Inventors: Richard L. Mueller JR., Jackson, WY (US); James Kermode, Los Altos, CA (US)

Correspondence Address: OLSON & HIERL, LTD., 36th Floor, 20 North Wacker Drive, Chicago, IL, 60606, US

(21) Appl. No.: 10/423,171

(22) Filed: Apr. 25, 2003

(51) Int. Cl. A61B 5/00

(52) U.S. Cl. 600/573

(57) ABSTRACT

A breast exudate expression device includes a frame and a pair of opposed, elongate compression members attached to the frame and spaced from one another. The compression members are mounted to the frame for movement toward one another from an open position to a constriction position. The compression members are maintained in opposing relationship to one another by the frame and are adapted to receive a human breast nipple therebetween. The compression members express a fluid aliquot from the nipple for use in evaluating, diagnosing, and managing breast diseases. A method of collecting a breast exudate utilizing a device as well as kits for collecting a breast exudate are also described.

### TECHNICAL FIELD OF THE INVENTION

This invention relates generally to devices and methods for collecting fluid samples from human mammary ducts. More specifically, the invention relates to devices and methods for expressing mammary fluid and other breast exudates from the mammary glands of a human for evaluating, diagnosing, and managing breast diseases.

### BACKGROUND OF THE INVENTION

Breast cancer is one of the most common forms of cancer in women. A key to treatment is early detection. For example, annual mammograms have been recommended in hopes of early detection of breast cancer. One problem with mammographic imaging is that it can only detect breast cancer once it has taken tangible form as a tumor. All too often, breast cancer is discovered at a stage that is too far advanced, when therapeutic options and survival rates are severely limited. Therefore, more sensitive and reliable methods and devices are needed to detect cancerous, pre-cancerous, and other cancer indicators of the breast at an early stage. Such methods and devices could significantly improve breast cancer survival. While breast cancer is most common among women, in rare instances breast cancer can occur in men.

A vast majority of breast cancers reportedly begin in the lining of mammary ducts. Studies have indicated that fluid within the mammary ducts can contain high levels of breast cancer markers, and that an estimated 80% to 90% of all breast cancers occur within the intraductal epithelium of the mammary glands. The fluid within the breast ducts contains an assemblage and concentration of hormones, growth factors and other potential markers comparable to those secreted by, or acting upon, the surrounding cells of the alveolar-ductal system. Mammary fluid also typically contains cells and solid cellular debris or products that can be used in cytological or immunological assays for breast cancer.

An early method of detecting breast cancer based on analysis of mammary fluid involves analyzing a sample of mammary fluid that was excreted by the mammary ducts without outside intervention. In other words, if a mammary duct is actively discharging a fluid, this fluid is analyzed. Often breasts do not discharge fluid spontaneously, even though breast cancer markers may be present in the mammary fluid. There is, therefore, a need for more widely applicable, methods and devices to obtain samples of mammary fluid or other breast exudate for use in evaluating, diagnosing and managing breast disease including cancer. Such methods and devices should be simple enough to be utilized by a physician, or more preferably by a nurse, physician's assistant, or technician during a visit to a physician's office.

One procedure based on the analysis of mammary fluid is called ductal lavage. This method entails introducing saline into the mammary ducts via a small catheter and removing the introduced saline solution from the mammary ducts by vacuum. The recovered fluid is then subjected to immunological or cytological evaluation for the presence of breast cancer markers. This is a delicate procedure requiring considerable skill on the part of the clinician performing the catheterization. One advantage of such methods is that fluid samples can be retrieved from individual ducts, thus facilitating location of the cancerous area within the breast. One difficulty with such procedures is that they involve introduction of a catheter into the very small ducts in the breast nipple.

Another method that has been developed is taught in U.S. Pat. No. 6,328,709 to Hung et. al., which describes a device for aspirating and retrieving breast duct fluid. The device described by Hung et al. uses a vacuum to cause the breast nipple to exude the fluid in a manner similar to breast pumps utilized by lactating women to collect breast milk. Similar to ductal lavage, the method and device described by Hung et al. requires costly equipment and specialized knowledge.

U.S. Pat. No. 6,471,660 to Covington describes a method of manually expressing a breast exudate that involves finger manipulation of the breast by the patient or a clinician. While this method can be cost effective, manipulation of the breast by finger pressure, as described by Covington, is only marginally effective for expressing fluid from a mammary duct.

The present invention provides a device and method for expressing a breast exudate from a breast nipple, which method overcomes many of the shortcomings of these prior methods and devices.

### SUMMARY OF THE INVENTION

A breast exudate expression device of the present invention includes a frame and a pair of opposed, elongate compression members attached to the frame. The compression members are mounted to the frame for movement toward one another from an open position to a constriction position. The compression members are maintained in opposed relationship to one another by the frame and are adapted to engage and constrict a human breast nipple between the compression members. Preferably the frame comprises a pair of opposed housings spaced from one another, each housing having a compression member mounted thereto. More preferably, the frame comprises a pair of opposed arms held in substantially parallel, spaced relationship to one another by a common base, each arm having a distal end portion having a compression member, such as a roller, mounted thereto.

In use, the device is positioned about a human breast nipple while in open position, and the compression members are urged toward one another into a constriction position so as to compress the nipple. The device is positioned so that the region posterior to the sphincter muscles in the nipple is compressed, thus forcing fluid outwardly (anteriorly) through the orifice of the duct. A sample of visible exudate can then be gathered into a collection vessel such as a capillary tube, an absorbent pad, and the like, for analysis. The clinician retrieving the sample can note the duct in the nipple from which the exudate was obtained, thus facilitating location of any cancerous regions within the breast.

The compression members of the device can be configured to provide greater, more localized pressure on the mammary ducts than can be achieved through finger manipulation of the breast or its nipple. The localization of applied pressure increases the efficiency of expression of the exudate (e.g., increasing the volume of exudate) relative to finger manipulation methods. In addition, the device and method of the present invention can be performed in a doctor's office without catheterization of the nipple, a procedure required for ductal lavage. The device is relatively easy to use, such that a nurse practitioner, a nurse, a physician's assistant, or a technician can utilize the device to express a fluid sample from a patient for analysis.

In one preferred embodiment the compression members are elongate rollers rotatably attached to the frame. Preferably, the frame includes two parallel, opposed housings, and the rollers are mounted in the respective housings, partially protruding therefrom. The rollers are maintained in substantially parallel opposed relationship to one another by resilient members attached to the housings. The rollers are spaced from one another so that in open position, substantially the entire areola of the breast can be engaged therebetween.

Preferably the rollers are cylindrical, having a longitudinal axis, and are symmetrically disposed about an axle or other rotatable mounting. Each roller is rotatably mounted to a portion of a housing. The rollers can be mounted so that they rotate about their longitudinal axis. Alternatively, one or both of the rollers can be mounted so that the axis of rotation is offset from the longitudinal axis of the roller providing a eccentric rotational motion.

Optionally, one or both of the rollers can be associated with a rack and pinion mechanism for rotating the rollers as they are reciprocated from open position to constriction position. In such embodiments, the roller mounting preferably includes a pinion movably engaged with a rack attached to an opposed housing. As the housings and their associated rollers are reciprocated toward one another, the movement of the racks relative to the pinions caused the pinions to rotate, which in turn rotates the rollers. Rotation of the rollers is biased to exert a forward (i.e., anteriorly-directed) force on the sinus of the mammary duct when the areola of a human breast is engaged between the rollers and the rollers are urged from open position into constriction position. The constricting force on the sinus due to compression from the rollers, in combination with the anteriorly-directed force on the sinus due to the rotation of the rollers results in a “milking action” that urges fluid within the duct to exude from the duct orifice.

In another preferred embodiment, the compression members are a pair of elongate blocks or “anvils” having opposed, elongate, substantially flat, truncate breast-contacting surfaces. The anvils are preferably pivotally attached to the frame, preferably with the breast-contacting surfaces of the anvils in non-parallel relationship to each other when the anvils are in open position. The edges of the anvil are preferably blunt or rounded off. The non-parallel breast-contacting surfaces are preferably configured such that when the areola of a human breast is engaged between the breast-contacting surfaces of the anvils, at least one of the anvils will pivot as the device is urged from open position to constriction position. The pivoting anvil(s) exert an anteriorly-directed force on the sinus of a mammary duct as the areola is constricted, to provide a “milking action” to urge mammary fluid out of the duct orifice. Preferably both anvils pivot; however, in some embodiments only one anvil pivots and the other remains stationary.

In yet another preferred embodiment the compression members are a pair of blunt edged blades mounted to a frame. Preferably the blades are elongate, substantially wedge-shaped, and are constructed from a flexible, resilient material. The working edge (i.e., the apex) of the blade is blunt, and the relatively broader base of the elongate blade is affixed to opposing flat, elongate surfaces of the frame. Preferably, the paired blades are maintained in opposed relationship to one another by the frame. In some preferred embodiments, the frame is composed of two elongate housing portions having substantially flat, movable blade-mounting surfaces held in opposed, parallel relationship to one another by resilient connecting members, such as resilient bands, springs, and the like. The bases of the wedge-shaped blades are affixed to the mounting surfaces of the housings so that the blunt, working edges of the blades are maintained in opposed, parallel relationship to one another. Alternatively, the blades can be pivotally mounted to the housings.

The blunt, working edges of the blades are spaced from one another by a distance sufficient to engage a human breast nipple therebetween without significant constriction of the nipple when the blades are in open position. Devices having varying degrees of blade spacing in open position can be provided to accommodate different sized breast nipples. Preferably, the spacing of the working edges of the blades in open position is sufficient to engage substantially the entire areola of the breast therebetween.

Preferably the blunt edges of the wedge-shaped blades are flexible and are each offset from the midline of the base in the same direction so that when the areola of a human breast is engaged between the blades and the blades are urged into constriction position the blunt edges of the blades flex in the direction of the offset to provide a slight anterior (forward) force on the areola, which facilitates expression of fluid from the orifice of the nipple. Preferably the constriction of the areola occurs slightly posterior to the lactiferous sinus and sphincter of the duct. Fluid within the sinus of the duct, posterior to the sphincter, is forced in an anterior direction toward the orifice of the nipple duct by the anteriorly-directed force exerted by the flexing blades. In this way, repeated compression and release of the areola creates a “milking action” to facilitate expression of fluids from the duct.

In another preferred embodiment, the breast exudate expression devices of the present invention consist of two complementary modules. The modules can be provided in assembled or disassembled form. The devices of the invention can also be provided in a packaged kit including instructional material describing assembly and use of the devices for evaluating, diagnosing, and managing breast diseases.

Additionally, the present invention encompasses a method of expressing an exudate from a human breast utilizing the above-described devices.

### BRIEF DESCRIPTION OF THE DRAWINGS

In the drawings,

FIG. 1 is an exploded perspective view of breast exudate expression device 100, wherein the compression members are rollers;

FIG. 2A illustrates device 100 of FIG. 1, having a collapsible frame, fully assembled in open position; FIG. 2B illustrates device 100 in constriction position with the frame collapsed;

FIG. 3A illustrates a rack and pinion mechanism having a pinion axially affixed to a roller, the pinion being operably engagable with a rack affixed to an opposed housing; FIG. 3B illustrates an alternative rack and pinion configuration;

FIG. 4 is a perspective view of breast exudate expression device 200, wherein the compression members are anvils;

FIG. 5 is a sectional detail of an anvil of the device 200 shown in FIG. 4;

FIG. 6 is a perspective view of module 202 of device 200 of FIG. 4;

FIG. 7A illustrates of the anvils of device 200 in open position with a human breast nipple engaged therebetween; FIG. 7B illustrates of the anvils of device 200 in a constriction position with a human breast nipple engaged therebetween;

FIG. 8 illustrates breast exudate expression device 300, wherein the compression members are flexible, wedge-shaped blades;

FIG. 9 is a cross-sectional view of device 300 of FIG. 8, taken through plane 9-9;

FIG. 10 illustrates breast exudate expression device 400, wherein the frame comprises a pair of arms held in substantially parallel, spaced relationship to one another by a common base, each arm having a roller, rotatably mounted perpendicular to the arm;

FIG. 11 illustrates a plan view of device 400;

FIG. 12 illustrates breast exudate expression device 500, wherein the frame comprises a pair of arms held in substantially parallel, spaced relationship to one another by a flexible, resilient common base, each arm having a roller, rotatably mounted thereto; and

FIG. 13 illustrates breast exudate expression device 600, wherein the frame comprises a pair of arms held in substantially parallel, spaced relationship to one another by a common base, each arm having a roller, rotatably mounted substantially coplanar with the arm.

### DETAILED DESCRIPTION OF PREFERRED EMBODIMENTS

A breast exudate expression device of the present invention includes a frame and a pair of opposed, elongate compression members attached to the frame. The compression members are adapted to engage and constrict a human breast nipple therebetween and are mounted to the frame for movement toward one another from an open position to a constriction position. Preferably the compression members reciprocate toward one another.

Preferably the compression members have a transverse (i.e., perpendicular to the longitudinal axis thereof) cross-sectional dimension that is less than the diameter of an adult human finger (i.e., no more than about 20 millimeters). More preferably the transverse cross-sectional dimension of the compression members is not more than about 10 millimeters, most preferably not more than about 5 millimeters, in the region of the compression member that contacts the breast. With compression members so dimensioned, the devices of the present invention offer a distinct advantage over techniques involving the use of finger manipulation of the breast. The narrower transverse cross-section of the compression members allows the clinician to apply a more localized pressure on the mammary ducts, closer to the sphincter than can be achieved with finger manipulation. The relatively narrow dimension of the compression members improves the efficiency of inducing the ductal fluid to exude from the duct.

The frame can comprise a pair of opposed housings attachable to one another by flexible members such as resilient bands or strips. The flexible members maintain the housings aligned in opposed relationship to one another. Alternatively, the housings can be maintained in opposed relationship to one another by guide-pins attached to a housing at one end and slidably engaging a socket in an opposed housing. In a particularly preferred embodiment, the housings are maintained in opposed relationship to one another by guide-pins and the housings are biased in an open position by one or more springs or resilient bands.

The housings are adapted to receive a compression member therein or to mount a compression member thereon. Compression members are mounted in or on the housings so that the compression members are maintained in opposed relationship to one another.

The housings and other frame members can be constructed from any convenient material, such as a plastic, metal, or a combination thereof. Suitable plastics include polyolefins such as polystyrene, polyethylene, polypropylene, and the like; polyesters such as polyethylene terephthalate (PET), and the like; polyamides such as nylons, and the like; polyimides; polyurethanes; halogenated polyolefins such as polyvinyl chloride, polytetrafluorethylene, and the like; and like polymeric materials of construction. Suitable metals include aluminum, stainless steel, enameled mild steel, chrome or nickel plated steel, titanium alloys, magnesium alloys, and the like. Preferably the frame is constructed from a light weight plastic material.

In a particularly preferred embodiment, the frame comprises a pair of arms held in substantially parallel, spaced relationship to one another by a common base, each arm having a distal end portion with a compression member mounted thereto. Preferably, either the base or the arms, or both, are constructed of resilient materials to facilitate movement of the compression members toward one another. Optionally, the base can be fluted or crenellated to afford flexibility and resilience to the base, so that the base is compressed when a compression force is applied thereto. Alternatively, the base can be made of a block of compressible material such as polyurethane foam, and the like. When the compression force applied to the base is released, the resilient nature of the base causes it to return to its original configuration. A resilient, compressible base facilitates movement or reciprocation of the opposed compression members toward each other form open position to constriction position while a human breast nipple is received and held between the compression members.

In preferred embodiments of the present invention the compression members are elongate rollers rotatably mounted to the frame. Preferably the rollers are cylindrical, having a longitudinal axis, and are symmetrically disposed about an axle or other rotatable mounting. Each roller is rotatably mounted to a portion of the frame. The rollers can be mounted so that they rotate about their longitudinal axis. Alternatively, one or both of the rollers can be mounted so that the axis of rotation is offset from the longitudinal axis of the roller. The rollers can optionally have an asymmetric cross-sectional profile. For example the cross-section can be elliptical, ovoid, tear-shaped, and the like.

The rollers are preferably constructed from light weight materials such as nylon, high density polyethylene, polystyrene, natural rubber, synthetic rubber, silicone rubber, and the like. Preferably the rollers are substantially non-resilient.

In other preferred embodiments, the compression members are a pair of elongate blocks or “anvils” having opposed, elongate, substantially flat, breast-contacting surfaces having blunt, rounded (i.e. truncate) edges. The anvils are preferably pivotally attached to the frame, preferably such that the breast-contacting surfaces of the anvils are non-parallel when the anvils are in open position. The non-parallel breast-contacting surfaces are preferably configured such that when the areola of a human breast is engaged between the anvils, at least one of the anvils will pivot as the anvils are urged from open position to constriction position. The pivoting anvil(s) thereby exert an anteriorly-directed force on the sinus of a mammary duct as the areola is constricted, to provide a milking action to urge mammary fluid out of the duct orifice. Preferably both anvils pivot, however, in some embodiments one anvil pivots and the other remains stationary.

The anvils are preferably composed of light weight, non-resilient materials such as nylon, high density polyethylene, polystyrene, and the like.

In another preferred embodiment the compression members are a pair of blunt edged blades. Preferably the blades are elongate, substantially wedge-shaped, and are constructed from a flexible, resilient material. Preferably the blades are made from resilient polymeric materials such as natural or synthetic rubbers, a polyurethane, and the like.

Preferably the working edge (i.e., the apex) of the blade is blunt, and the relatively broader base of the blade is affixed to opposing flat, elongate surfaces of the frame. In some preferred embodiments the blades are mounted to substantially flat blade-mounting surfaces of the frame held in opposed, parallel relationship to one another. The bases of the wedge-shaped blades are affixed to the mounting surfaces so that the blunt, working edges of the blades are maintained in opposed, parallel relationship to one another. Alternatively, the blades can be pivotally mounted to the frame so that the blades will pivot when they are reciprocated toward one another with a breast nipple engaged therebetween.

The blunt, working edges of the blades are spaced from one another by a distance sufficient to engage a human breast nipple therebetween without significant constriction of the nipple when the blades are in open position. The devices can be provided in sizes or grades having varying degrees of blade spacing in open position to accommodate different sized breast nipples. Preferably, the spacing of the working edges of the blades in open position will be sufficient to engage substantially the entire areola of a patient's breast therebetween.

Preferably the blunt edges of the wedge-shaped blades are flexible and are each offset from the midline of the base in the same direction so that when the areola of a human breast is engaged between the blades and the blades are urged into constriction position the blunt edges of the blades flex in the direction of the offset. When received therebetween, the nipple is constricted posterior to the lactiferous sinus and sphincter of a mammary duct. Fluid within the sinus of the duct, posterior to the sphincter, is forced in an anterior direction toward the orifice of the duct by the anteriorly-directed force exerted by the flexing blades. Repeated constriction and release of the nipple provides a milking action, which facilitates expression of fluid from the orifice of the nipple.

Another aspect of the present invention is a packaged kit containing at least one breast exudate expression device of the present invention, an instructional indicia, and optionally an exudate collection vessel. The instructional indicia can include a description of the device, instructions for assembling the device, instructions for using the device to express a fluid sample from a human breast, instructions for collecting an expressed fluid sample, or any combination thereof. The instructional materials can be provided in the form of a label, a printed pamphlet, a video tape, a DVD, a CDROM, a DVDROM, or a combination thereof, and the like. The collection vessel can be a capillary tube, a sealable vial, an absorbent collection paper, a pipette, and a combination thereof, for collecting an expressed fluid aliquot from the breast nipple. The breast exudate expression device can be modular and can be included in the packaging material in a disassembled or partially disassembled state. Alternatively, the device can be provided in a fully assembled form.

Another aspect of the present invention is a method for collecting a sample of an exudate from a human breast nipple. As used herein and in the appended claims, the term “breast nipple” and grammatical variations thereof refers to the entire areolar region of the breast including the areola and the protruding portion thereof commonly referred to as the “nipple.” The method involves engaging a human breast nipple between the compression members of a breast exudate expression device of the invention with the compression members in open position. The compression members are positioned over the nipple by a clinician so that they are posterior to the sphincter of a mammary duct. The compression members are then urged into constriction position to exert a compression force on the mammary duct posterior to the sphincter. The compression force causes fluid within the duct to exude from the duct orifice. A sample of the fluid can then be collected with a capillary tube, absorbent sample collection paper, pipette, vial, and the like. The sample can then be submitted to a clinical laboratory for cytological or immunological analysis for breast cancer markers or other indications of breast disease. The clinician can also note the location of the duct from which the fluid sample was collected to aid in locating the source of any breast cancer markers detected in the fluid.

Optionally, forceps or tongs adapted to hold the device can be used to facilitate manipulation of the device over the nipple and reciprocation thereof, if desired. Such forceps provide addition leverage and can be particularly advantageous when attempting to manipulate the device over a relatively small nipple.

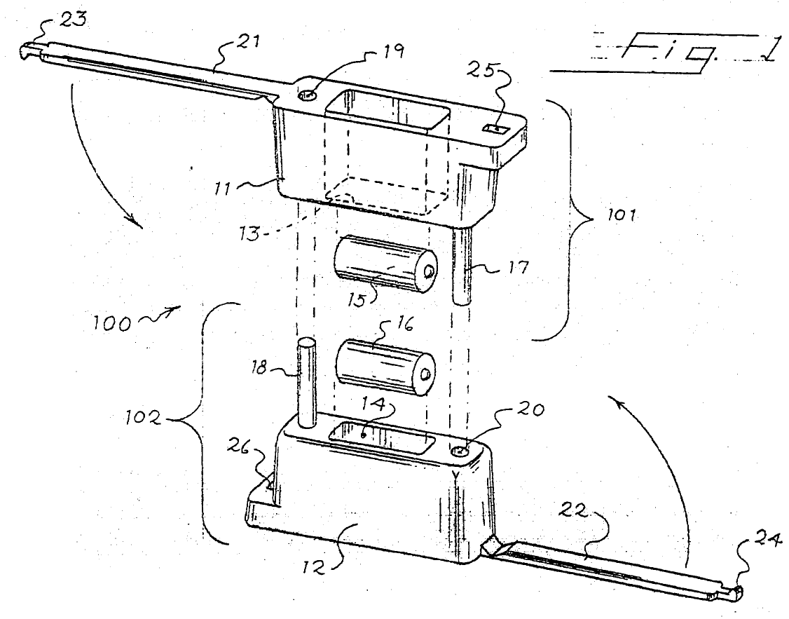

Turning now to the Drawings, FIG. 1 is an exploded perspective view of a preferred breast exudate expression device of the invention. Device 100 includes a pair of opposed but co-acting housings 11,12, each defining a recess 13,14 for receiving a respective roller 15,16 therein. Each housing 11,12 also includes a guide-pin 17,18 on one side of the housing and defines a complementary socket 19,20 on the side of the housing opposite guide-pin 17,18. Each guide-pin 17,18 is positioned and sized to engage socket 19,20 of its opposed housing 11,12. A flexible member 21,22 is hingedly attached at its proximal end to the respective side of housing 11,12 opposite guide-pin 17,18. Each flexible member 21,22 includes latch portion 23,24 at its distal end. Each housing 11,12 also defines a slot 25,26 on the side of the housing adjacent to guide-pin 17,18, which slot is adapted to engage latch portion 23,24 of corresponding opposed flexible member 21,22.

Each housing 11,12, with its associated roller 15,16, guide-pin 17,18, and flexible member 21,22 constitutes a module 101,102. The two modules 101,102 can be assembled into working device 100 by first inserting a guide-pin 17 of module 101 into socket 20 of module 102 and inserting guide-pin 18 of module 102 into socket 19 of module 101. To complete the assembly, latch 23 of flexible member 21 is inserted into slot 26 of housing 12, and latch 24 of flexible member 22 is inserted into slot 25 of housing 11.

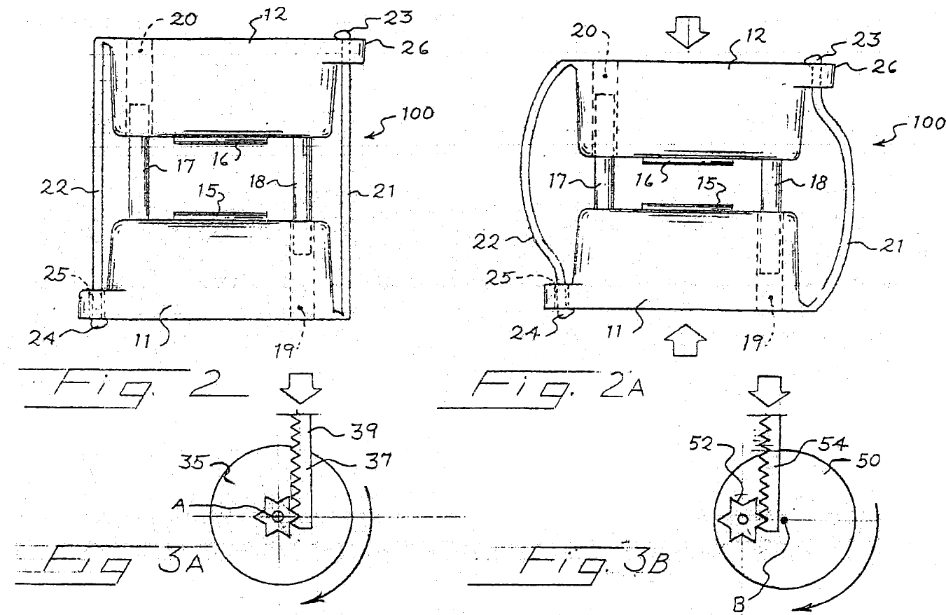

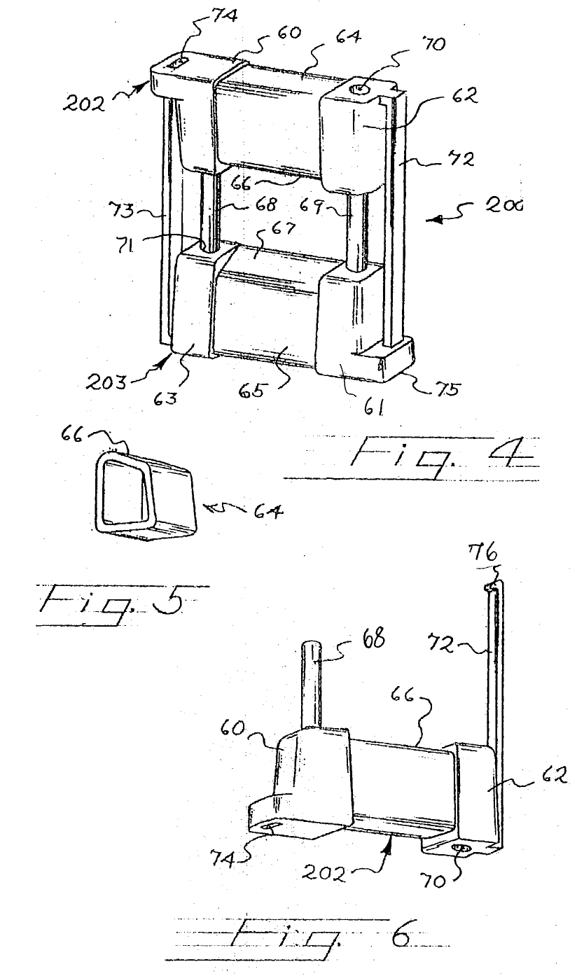

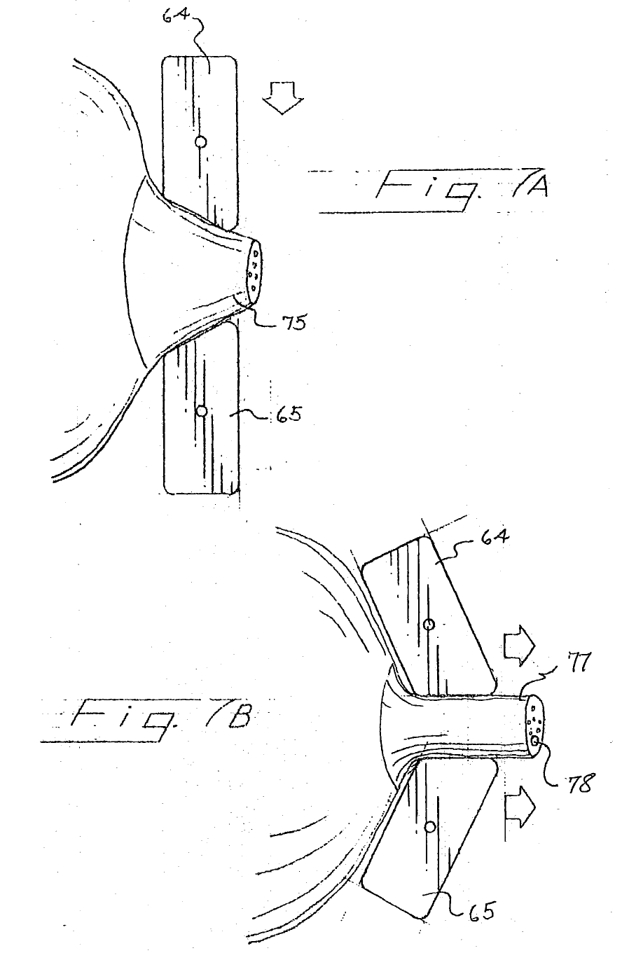

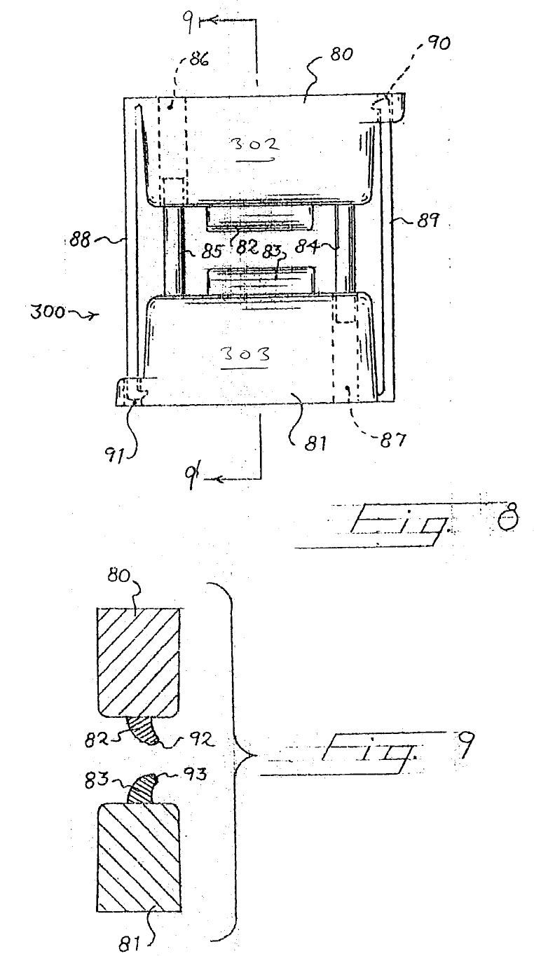

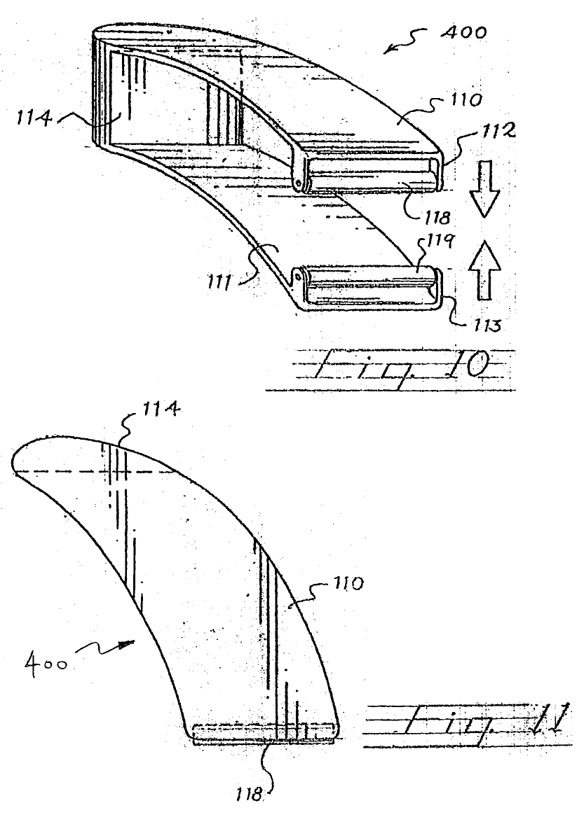

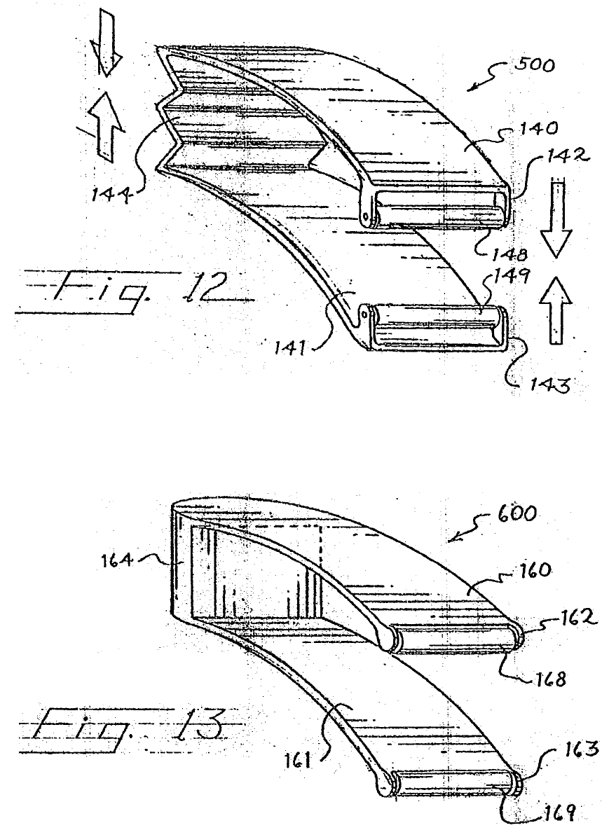

FIGS. 2A and 2B illustrate device 100 fully assembled for use. Housings 11,12, guide-pins 17,18 and flexible members 21,22 together define a collapsible frame. As seen in FIG. 2A, flexible members 21,22 act as leaf springs to bias housings 11,12 and their associated rollers 15,16 in open position, as shown. FIG. 2B illustrates device 100 in a collapsed, constriction position. Application of opposing forces to housings 11,12, to reciprocate the opposed housings toward one another causes flexible members 21,22 to bow outwardly and the gap between rollers 15,16 to decrease.

Optionally, the breast exudate expression device of the invention can include a rack and pinion mechanism for rotating the rollers as the housings are reciprocated toward each other from open position to constriction position. Each roller can include a pinion axially affixed to one of its ends. The pinion is operably engagable with a rack that is affixed to an opposed housing. Reciprocation of the housings (and their attached rollers) toward one another causes the rack to engage the pinion, thus causing the pinion and its associated roller to rotate.

FIG. 3A is a side view of a roller 35 and associated pinion 37 within housing 12 illustrating how the pinion 37 engages with a rack 39. In this embodiment, pinion 37 is centered on the longitudinal axis of and mounted to one end of roller 35. When rack 39 is moved relative to roller 35, pinion 37 rotates, causing the attached roller 35 to rotate about its longitudinal axis A. The rotation of the roller 35 creates a wringing effect on a breast nipple engaged between a pair of such rollers as the rotating rollers are reciprocated toward one another from open position to constriction position. This wringing action is beneficial for urging out any fluid present in the ducts.

FIG. 3B is a side view of an alternative roller arrangement. A roller 50 includes a pinion 52 eccentrically mounted to one end of roller 50, offset from the longitudinal axis B of the roller. Pinion 52 is engaged with rack 54. When rack 54 is moved relative to roller 50, pinion 52 turns, which causes attached roller 50 to rotate eccentrically. Such an eccentric rack and pinion arrangement provides a combination of squeezing and wringing forces on a breast nipple received between eccentric rollers. The squeezing force provides a differential pumping action of the ducts within the nipple, somewhat analogous in action to a peristaltic pump.

FIG. 4 illustrates another preferred embodiment of the present invention. The compression members in this particular embodiment are anvils having non-parallel breast contacting surfaces. Device 200 has a collapsible frame that includes paired frame members 60,62 and 61,63, having respective anvils 64,65 with breast contacting surfaces 66,67 pivotally mounted therebetween. Frame member 60 includes a guide-pin 68, which is adapted to engage a socket 71 defined by frame member 63. Corresponding guide-pin 69 of frame member 61 is received in socket 70 of frame member 62. Flexible member 72 is hingedly attached at its proximal end to frame member 62. The distal end of flexible member 72 is provided with latch 76 (FIG. 6) adapted to engage a slot 75 defined by frame member 61. Flexible member 73 is similarly connected to frame member 63 and is adapted to engage slot 74 in frame member 60. Flexible members 72,73 function in a similar manner to flexible members 21,22 in device 100, as described above. Anvils 64,65 are maintained in opposed relationship to each other by their associated paired frame members 60,62 and 61,63 and guide-pins 68,69. Breast contacting surfaces 66 and 67 are disposed in non-parallel relationship to each other and define intersecting planes when at rest by virtue of the asymmetric shape of the anvils, shown in more detail in FIG. 5.

Anvils 64,65 can be fixed, but are preferably pivotally mounted between paired frame members 60,62 and 61,63, respectively, so that the breast contacting surfaces 66,67 of each anvil can pivot into substantially parallel relationship to one another when reciprocated from open position (shown in FIG. 4) to constriction position with a breast nipple engaged between the breast contacting surfaces of the anvils. In use, frame member 61 is in opposed relationship to frame member 62 and frame member 60 is in opposed relationship to frame member 63.

Each assembly of frame members 60,62 and 61,63 with their associated anvil 64,65, guide-pin 68,69, and flexible member 72,73 constitute modules 202,203. Device 200 is assembled from modules 202 and 203 in substantially the same manner as described for device 100 above.

FIG. 6 illustrates a perspective view of individual module 202 of device 200. As shown in FIG. 6, flexible member 72 includes a latch portion 76 at its distal end. Housing member 60 defines a slot 74 adapted to engage a latch from module 203 (not shown) similar to latch 76. Housing member 62 defines a socket 70 for receiving the guide-pin 69 from module 203 (not shown).

FIGS. 7A and 7B illustrate the anvils 64 and 65 of device 200 with a human breast nipple 77 engaged therebetween. As the anvils are reciprocated toward one another from open position (FIG. 7A), the initially non-parallel breast contacting surfaces 66 and 67, respectively, of anvils 64 and 65 pivot to a substantially parallel relationship to one another as the anvils move to constriction position (FIG. 7B). The pivoting of anvils 64 and 65, while constricting the ducts within the nipple 77, also exerts an anteriorly directed force on the ducts to push any fluid within the ducts out through the duct orifice to form a droplet 78, which can be collected for analysis. The anvils can be spring biased, if desired.

FIG. 8 illustrates yet another preferred embodiment of the breast exudate expression device of the invention, wherein the compression members are flexible, elongate, wedge-shaped blades. Device 300 is composed of two modules 302 and 303. Module 302 includes a housing 80, having a flexible, elongate, substantially wedge-shaped blade 82, affixed thereto. Likewise, module 303 includes housing 81 having a flexible, elongate, substantially wedge shaped blade 83 affixed thereto. Housings 80 and 81 and their respective associated blades 82 and 83 are maintained in a substantially parallel, opposed relationship to one another by guide-pins 84 and 85, which are received by corresponding sockets 86 and 87 defined by housings 80 and 81, respectively. Flexible spring members 88 and 89 are hingedly attached to housings 80 and 81 and are adapted to engage slots 90 and 91 in housings 80 and 81, respectively.

FIG. 9 is a cross-sectional view of device 300 through plane 9-9 in FIG. 8 and showing the substantially wedge-shaped profile of blades 82,83. As shown in FIG. 9, the blades are preferably curved and have blunt edges 92,93 to minimize likelihood of trauma to a breast nipple engaged therebetween. Preferably the curvatures of blades 82,83 are both biased in the same direction, so that as the blades are urged from open position to constriction position with a breast nipple engaged therebetween, the blades both flex in the same direction to exert an anteriorly-directed force on the nipple ducts.

FIG. 10 illustrates a particularly preferred embodiment of the device of the present invention. Device 400 includes a pair of opposed flexible, lingulate arms 110,111, the proximal end of each arm integral with base 114, which maintains arms 110,111 in a spaced, substantially parallel relationship to one another. Base 114 can be rigid or compressible. Arms 110,111 are provided at the distal end portions thereof with lugs 112,113, perpendicular to arms 110,111, to which rollers 118,119 are rotatably mounted. In use, a breast nipple is engaged between rollers 118,119 which have an outside diameter of about 5 mm. A compression force is then applied to arms 110,111, which causes rollers 118,119 to move toward each other and constrict the nipple therebetween. Once the nipple is constricted, rollers 118,119 can be moved anteriorly along the nipple to force fluid from a duct.

FIG. 11 is a plan view of device 400, showing the lingular shape of arms 110,111.

FIG. 12 illustrates another preferred embodiment of the device shown in FIG. 10, wherein the base is compressible. Device 500 includes a pair of opposed lingulate arms 140,141, the proximal end of each arm being mounted to a common base 144, which maintains arms 140,141 in a spaced, substantially parallel relationship to one another. Arms 140,141 each have lugs 142,143 at the respective distal end portions thereof to which rollers 148,149 are rotatably mounted. Base 144 is fluted and resilient so that it can be compressed when a compression force is applied thereto. Upon release of the compression force, the base resumes its original, non-compressed or relaxed configuration.

FIG. 13 illustrates yet another preferred embodiment of the device of the present invention. Device 600 includes a pair of opposed lingulate arms 160,161, the proximal end of each arm being mounted to base 164 which maintains arms 160,161 in a spaced, substantially parallel relationship to one another. Arms 160,161 each have a distal end portion 162,163 to which rollers 168,169 are rotatably mounted with the longitudinal axes thereof substantially coplanar with the planes of arms 160,161. In use, a breast nipple is received between rollers 168,169. A compression force is then applied to arms 160,161, which causes rollers 168,169 to moved toward each other to constrict the nipple engaged therebetween. Once the nipple is constricted, rollers 168,169 can be moved anteriorly along the nipple to force fluid from a duct.

The devices of the present invention, as described above, can provide greater, more localized pressure on the mammary ducts than can be achieved through finger manipulation of the breasts. The localization of pressure increases the efficiency of expression of the exudate (e.g., increasing the volume of exudate) relative to finger manipulation methods. In addition, the devices and methods of the present invention can be performed in a doctor's office without catheterization of the nipple, which is required for ductal lavage. The device is relatively easy to use, such that a nurse practitioner, a nurse, a physician's assistant, or a technician can utilize the device to express a fluid sample from a patient for analysis and diagnosis.

The foregoing description is to be taken as illustrative, but not limiting. Still other variants within the spirit and scope of the present invention will readily present themselves to those skilled in the art.

We claim:

1. A breast exudate expression device comprising:
a frame; and
a pair of opposed, elongate compression members spaced from one another, adapted to engage a human breast nipple therebetween, and mounted to the frame for movement toward one another from an open position to a constriction position.

2. The device of claim 1 wherein the compression members are mounted for reciprocation toward one another.

3. The device of claim 1 wherein the compression members are elongate, cylindrical rollers, each having a longitudinal axis, and each being rotatably mounted to the frame.

4. The device of claim 3 wherein each of the rollers is symmetrically mounted to the frame such that the axis of rotation of the roller coincides with the longitudinal axis of the roller.

5. The device of claim 3 wherein at least one of the rollers is asymmetrically mounted to the frame such that the axis of rotation of the roller is offset from the longitudinal axis of the roller.

6. The device of claim 3 further comprising a rack and pinion mechanism engaged with the rollers, such that the rollers rotate as the rollers are reciprocated from open position to constriction position.

7. The device of claim 3 wherein the frame comprises a pair of opposed housings, each housing partially enclosing a roller, the housings being attachable to one another by resilient members adapted to maintain the housings in opposed relationship to each other.

8. The device of claim 3 wherein the frame comprises a pair of arms held in substantially parallel, spaced relationship to one another by a common base, each arm having a distal end portion with a compression member mounted thereto.

9. The device of claim 8 wherein the base is compressible.

10. The device of claim 8 wherein the compression members are rollers.

11. The device of claim 10 wherein the rollers are mounted substantially perpendicular to the arms.

12. The device of claim 10 wherein the rollers are mounted substantially coplanar with the arms.

13. The device of claim 1 wherein the compression members have a transverse cross-sectional dimension smaller than the diameter of an adult human finger.

14. The device of claim 13 wherein the transverse cross-sectional dimension of the compression members is not more than about 10 millimeters.

15. The device of claim 13 wherein the transverse cross-sectional dimension of the compression members is not more than about 5 millimeters.

16. The device of claim 1 wherein the compression members are blunt-edged, wedge-shaped blades.

17. The device of claim 16 wherein the blades are fixedly attached to the frame.

18. The device of claim 16 wherein the blades are pivotally attached to the frame.

19. The device of claim 16 wherein the blades are flexible.

20. The device of claim 16 wherein the frame comprises a pair of opposed housings, each housing having a blunt edged blade attached thereto such that the blades are maintained in opposed, substantially parallel relationship to one another, the housings being attachable to one another by resilient members adapted to maintain the housings in opposed relationship to each other.

21. The device of claim 1 wherein the compression members are anvils having opposed, substantially flat, truncate breast-contacting surfaces, the anvils being pivotally attached to the frame such that the breast contacting surfaces of the anvils are in non-parallel, opposed relationship to each other in open position and are adapted to pivot into substantially parallel relationship to each other in constricting position with a human breast nipple engaged therebetween.

22. The device of claim 1 wherein the compression members are biased in open position.

23. The device of claim 22 wherein the frame is resilient and the compression members are biased in open position by the resilient frame.

24. The device of claim 22 wherein the compression members are biased in open position by at least one spring interposed therebetween.

25. A breast exudate expression device comprising:
a collapsible frame including two opposed pairs of frame members each pair of frame members having a resilient member attached to an opposite frame member for maintaining the pairs of frame members in opposed relationship to and spaced from one another, for reciprocation of the pairs of frame members toward one another from an open position to a constriction position; and
two elongate compression members, one such compression member attached to each opposed pair of frame members and adapted to engage a human breast nipple therebetween.

26. The device of claim 25 wherein each pair of the paired frame members further includes at least one guide-pin for maintaining the compression members in substantially parallel, opposed relationship to one another.

27. The device of claim 25 wherein the compression members are anvils each pivotally mounted to a pair of frame members and having substantially flat, truncate breast-contacting surfaces, the breast contacting surfaces of the anvils being maintained in opposed relationship to one another by the paired frame members.

28. The device of claim 28 wherein the breast-contacting surfaces of the anvils are biased in non-parallel, opposed relationship to one another in open position and are adapted to pivot into substantially parallel opposed relationship to one another when in constriction position with a human breast nipple engaged therebetween.

29. A method for collecting a sample of an exudate from a human breast nipple and comprising the steps of:
engaging a human breast nipple between the compression members of a device of claim 1 with the compression members in open position, the compression members being positioned about the nipple and posterior to the sphincter of a mammary duct;
urging the compression members into constriction position so as to exert a compression force on the mammary duct posterior to the sphincter to cause fluid within the duct to exude from the duct orifice; and
collecting a sample of the so exuded fluid.

30. A kit comprising packaging material containing at least one a breast exudate expression device and instructional indicia; the device comprising a frame; and a pair of opposed, elongate compression members adapted to engage a human breast nipple therebetween, and mounted to the frame for movement toward one another from an open position to a constriction position.

31. The kit of claim 30 wherein the instructional indicia is selected from the group consisting of a description of the device, instructions for assembling the device, instructions for using the device to express a fluid sample from a human breast, instructions for collecting an expressed fluid sample, and a combination thereof.

32. The kit of claim 30 wherein the instructional indicia is in the form of a label, a printed pamphlet, a video tape, a DVD, a CDROM, a DVDROM, or a combination thereof.

33. The kit of claim 30 further comprising at least one collection vessel for gathering an expressed fluid aliquot from the breast nipple.

34. The kit of claim 33 wherein the collection vessel is selected from the group consisting of a capillary tube, a sealable vial, an absorbent collection paper, a pipette, and a combination thereof.

35. The kit of claim 30 wherein the device is modular and is present in the packaging material in a disassembled or partially disassembled state.
